(19) United States
(12) Patent Application Publication
(10) Pub. No.: US 2008/0095808 A1
(43) Pub. Date: Apr. 24, 2008

(54) Cosmetic candle

(76) Inventor: Erez Zabari, Rehonst (IL)

Correspondence Address: EDWIN TARVER, 16830 Ventura Blvd., SUITE 360, Encino, CA, 91436, US

(21) Appl. No.: 11/821,057

(22) Filed: Jun. 21, 2007

(30) Foreign Application Priority Data
Sep. 18, 2005 (IL) 170919

(51) Int. Cl. A61K 8/02; A61K 35/64; A61Q 19/00

(52) U.S. Cl. 424/401; 424/539

(57) ABSTRACT

A cosmetic candle comprising tiny grains embedded therein. The grains impart a mechanical property that can be exploited for scrubbing skin to peel off old or dead tissue with a concomitant massaging of molten waxes into the skin. Crushed pits and seeds of olives, apricots, peaches, citrus fruits and/or plums are natural products that can be used as grains in embodiments of the invention. In addition, solid salt grains, such as natural salts from the Dead Sea, or sugar grains, may also be used to that effect.

### CROSS-REFERENCE TO RELATED APPLICATION

None

This U.S. national stage application claims the benefit of the priority date of Israel patent application number 170919, filed on Sep. 18, 2005, and filed as PCT application number PCT/IL2006/001088 on Sep. 21, 2006.

### FEDERALLY SPONSORED RESEARCH

Not Applicable

### SEQUENCE LISTING OR PROGRAM

Not Applicable

### STATEMENT REGARDING COPYRIGHTED MATERIAL

Portions of the disclosure of this patent document contain material that is subject to copyright protection. The copyright owner has no objection to the facsimile reproduction by anyone of the patent document or the patent disclosure as it appears in the Patent and Trademark Office file or records, but otherwise reserves all copyright rights whatsoever.

### BACKGROUND

The present invention relates in general to fragranced candles and in particular to candles for cosmetic use.

Fragranced candles made of waxes and/or oils classified as cosmetic are common in the market place. Small amounts of warmed oil and/or wax can be directly massaged into the skin, leaving the skin smooth, soft and pleasantly scented. Therefore such candles not only appeal to the visual, tactile and olfactory senses but also can be used to treat the skin.

### SUMMARY

The present invention comprises a cosmetic candle containing tiny grains embedded therein. These grains impart a mechanical property that can be exploited for scrubbing the skin of a user to peel off old or dead tissue, with a concomitant massaging of molten waxes into the skin. Crushed pits and seeds of olives, apricots, peaches, citrus fruits and/or plums are natural products that can be used as grains in embodiments of the invention. Solid salt grains, such as natural salts from the Dead Sea, or sugar grains, may also be used. Grains of plastic resins such as polyethylene although suitable for the scrubbing activity, are potentially emitters of soot and unpleasant odors and therefore should not be included in the ingredients of such a candle.

### DESCRIPTION

The cosmetic candle of the present invention contains tiny grains embedded therein. These grains impart a mechanical property that can be exploited for scrubbing the skin of a user to peel off old or dead tissue, with a concomitant massaging of molten waxes into the skin. Crushed pits and seeds of olives, apricots, peaches, citrus fruits and/or plums are natural products that can be used as grains in embodiments of the invention. Solid salt grains, such as natural salts from the Dead Sea, or sugar grains, may also be used to that effect.

Grains of plastic resins such as polyethylene although suitable for the scrubbing activity, are potentially emitters of soot and unpleasant odors and therefore should not be included in the ingredients of such a candle.

According to the invention, suitable ingredients for making the matrix of the cosmetic candle are any waxes or paraffin waxes as known in the art of candle making. Additionally, any of the following are materials that can be employed in the making of the candles: palm kernel wax, beeswax, and propolis wax. Other ingredients possibly included, impart specific cleansing, aromatic, soothing and other cosmetic characteristics to the candles. Among these are chamomile extract, propylene glycol, aloe vera gel, papaya extract, mango extract, melon extract, vanilla planifolia extract, soybean flour, soy oil, carnauba wax, fragranced olive oil, jojoba oil, evening primrose oil, propylparaben, shea butter, honey, tocopheryl acetate, sweet almond oil, Dead Sea salts, citrus extracts such as extracts of orange, lemon, tangerine, lime and or grapefruit, and any combination thereof.

In accordance with a preferred embodiment of the present invention pits of olives and apricots are coarsely crushed and mixed with salts from the Dead Sea. This mixture is further crushed to prepare a fine powder consisting of tiny grains. The powder is further mixed with a selection of molten waxes and oils from the list mentioned above and heated to a specified temperature, such that a homogeneous mixture is formed. The candle is further formed as is in the prior art and is preferably disposed in a container made of a light metal, ceramic or glass. A plain wick, typically a hemp wick or any suitable wick without a metal core is affixed to the bottom of the container. The container provides for an aesthetic appeal element, while affording additional safety and convenience in handling the candle while lit.

What is claimed is:

1. A cosmetic candle having a matrix comprising:
a. grains embedded in the matrix for scrubbing a user's skin; and
b. materials contained in the matrix for massaging into a user's skin.

2. The cosmetic candle of claim 1, installed in a container, wherein a wick is affixed to a bottom of the container.

3. The cosmetic candle of claim 1, wherein the materials include at least one cosmetic ingredient selected from a group consisting of palm kernel wax, beeswax, propolis wax, propylene glycol, chamomile extract, aloe Vera gel, papaya extract, mango extract, melon extract, vanilla planifolia extract, soybean flour, soy oil, carnauba wax, fragranced olive oil, jojoba oil, evening primrose oil, propylparaben, Shea butter, honey, tocopheryl acetate, sweet almond oil, citrus extract, Dead Sea salts and any combination thereof.

4. The cosmetic candle of claim 3, wherein said citrus extract includes any member of a group of ingredients including, orange, lemon, tangerine, lime, grapefruit and any combination thereof.

5. The cosmetic candle of claim 1, wherein said grains comprise crushed pits and seeds of any item selected from a group of items consisting of apricots, olives, peaches, plums, citrus fruits and any combination thereof.

6. The cosmetic candle of claim 1, wherein said grains comprise any material selected from a group of materials consisting of solid salt, solid sugar, solid salts from the Dead Sea and any combination thereof.
